(19) United States
(12) Patent Application Publication
(10) Pub. No.: US 2006/0122177 A1
(43) Pub. Date: Jun. 8, 2006

(54) Phenylpiperazines with a combination of affinity for dopamine-D2 receptors and serotonin reuptake sites

(75) Inventors: Roelof van Hes, Weesp (NL); Pieter Smid, Weesp (NL); Cornelis G. Kruse, Weesp (NL); Martinus Th. M. Tulp, Weesp (NL)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: SOLVAY PHARMACEUTICALS B.V.

(21) Appl. No.: 11/294,611

(22) Filed: Dec. 6, 2005

Related U.S. Application Data
(60) Provisional application No. 60/633,449, filed on Dec. 7, 2004.

(51) Int. Cl. A61K 31/538; A61K 31/496; C07D 413/02; C07D 405/02; C07D 403/02

(52) U.S. Cl. 514/230.5; 544/105; 544/368; 544/375; 514/254.02; 514/254.11; 544/363; 514/253.06; 514/255.03; 544/396

(57) ABSTRACT

The invention relates to a group of novel phenylpiperazine derivatives with a dual mode of action: serotonin reuptake inhibition and affinity for dopamine-D₂ receptors and to methods for the preparation of these compounds. The invention also relates to the use of a compound disclosed herein for the manufacture of a medicament giving a beneficial effect.
The compounds have the general formula (1) 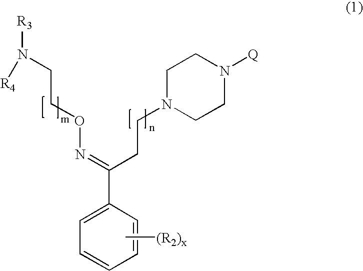 wherein the symbols have the meanings given in the specification.

The present invention relates to a group of novel phenylpiperazine derivatives with a dual mode of action: serotonin reuptake inhibition and affinity for dopamine-D₂ receptors and to methods for the preparation of these compounds. The invention also relates to the use of a compound disclosed herein for the manufacture of a medicament giving a beneficial effect. A beneficial effect is disclosed herein or apparent to a person skilled in the art from the specification and general knowledge in the art. The invention also relates to the use of a compound of the invention for the manufacture of a medicament for treating or preventing a disease or condition. More particularly, the invention relates to a new use for the treatment of a disease or condition disclosed herein or apparent to a person skilled in the art from the specification and general knowledge in the art. In embodiments of the invention specific compounds disclosed herein are used for the manufacture of a medicament useful in the treatment of disorders in which dopamine-D₂ receptors and serotonin reuptake sites are involved, or that can be treated via manipulation of those targets.

Phenylpiperazine derivatives with a dual action as dopamine-D₂ antagonists and serotonin reuptake inhibitors are known from WO 01/014330. This combination is useful for the treatment of schizophrenia and other psychotic disorders which enables a more complete treatment of all disease symptoms (e.g. positive symptoms and negative symptoms).

In patent specification GB 1 378 080 (1974) oxime derivatives of halophenyl piperazinyl-alkyl ketones have been disclosed that posses useful pharmacological activity, especially as analgesic agents, anti-inflammatory agents and musculotropic spasmolytic agents.

The goal of the present invention was to provide further compounds with a dual action as dopamine-D₂ antagonists and serotonin reuptake inhibitors.

The invention relates to compounds of the general formula (1): 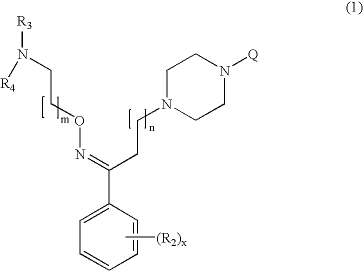
wherein:
m and n independently are either 1, 2, 3, 4, 5, 6, 7 or 8,
x is 0, 1, 2 or 3
R₂ is halogen, branched or unbranched alkyl(C₁₋₆), phenyl, benzyl, branched or unbranched alkoxy(C₁₋₆), trifluoromethyl or cyano
R₃ and R₄ independently represent hydrogen, alkyl(C₁₋₆), phenyl, benzyl or acetyl
group Q is chosen from structural fragments A-N 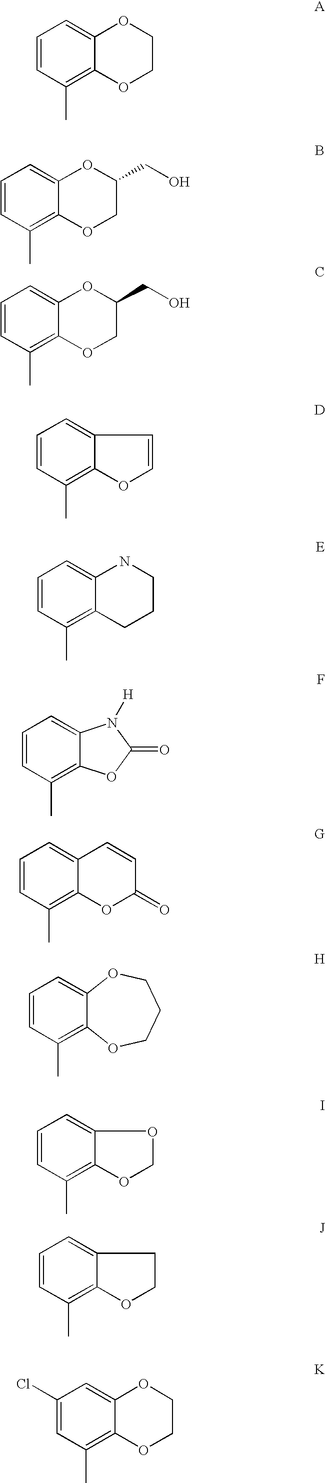 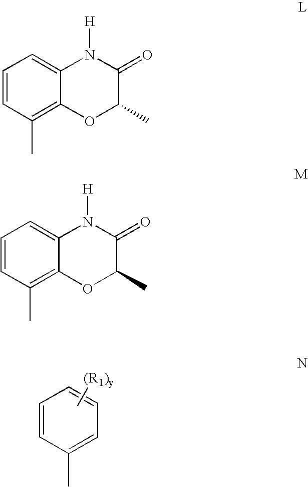
   wherein:
y is 1, 2 or 3
R₁ is halogen, branched or unbranched alkyl(C₁₋₆), phenyl, benzyl, branched or unbranched alkoxy(C₁₋₆), trifluoromethyl or cyano,
   and tautomers, stereoisomers and N-oxides thereof, as well as pharmacologically acceptable salts, hydrates and solvates of said compounds of formula (1) and its tautomers, stereoisomers and N-oxides.

Prodrugs of the compounds mentioned above are in the scope of the present invention. Prodrugs are therapeutic agents which are inactive per se but are transformed into one or more active metabolites. Prodrugs are bioreversible derivatives of drug molecules used to overcome some barriers to the utility of the parent drug molecule. These barriers include, but are not limited to, solubility, permeability, stability, presystemic metabolism and targeting limitations (Medicinal Chemistry: Principles and Practice, 1994, ISBN 0-85186-494-5, Ed.: F. D. King, p. 215; J. Stella, “Prodrugs as therapeutics”, Expert Opin. Ther. Patents, 14(3), 277-280, 2004; P. Ettmayer et al., “Lessons learned from marketed and investigational prodrugs”, J.Med.Chem., 47, 2393-2404, 2004). Pro-drugs, i.e. compounds which when administered to humans by any known route, are metabolised to compounds having formula (1), belong to the invention. In particular this relates to compounds with primary or secondary amino or hydroxy groups. Such compounds can be reacted with organic acids to yield compounds having formula (1) wherein an additional group is present which is easily removed after administration, for instance, but not limited to amidine, enamine, a Mannich base, a hydroxyl-methylene derivative, an O-(acyloxymethylene carbamate) derivative, carbamate, ester, amide or enaminone.

N-oxides of the compounds mentioned above are in the scope of the present invention. Tertiary amines may or may not give rise to N-oxide metabolites. The extend to what N-oxidation takes place varies from trace amounts to a near quantitative conversion. N-oxides may be more active than their corresponding tertiary amines or less active. Whilst N-oxides are easily reduced to their corresponding tertiary amines by chemical means, in the human body this happens to varying degrees. Some N-oxides undergo nearly quantitative reductive conversion to the corresponding tertiary amines, in other cases the conversion is a mere trace reaction or even completely absent. (M. H. Bickel: “The pharmacology and Biochemistry of N-oxides”, Pharmaco-logical Reviews, 21(4), 325-355, 1969).

Preferred compounds of the invention are compounds having formula (I) wherein m is 1, n is 2, 3, 4 or 5, x is 1, R₂ is 4-fluoro or 4-trifluoromethyl, R₃ and R4 independently represent hydrogen or methyl, group Q is chosen from structural fragments A, D, F or N, y is 1, and R₁ is branched or unbranched alkoxy(C₁₃), and tautomers, stereoisomers and N-oxides thereof, as well as pharmacologically acceptable salts, hydrates and solvates of said compounds of formula (1) and its tautomers, stereoisomers and N-oxides.

It has been found that the compounds according to the invention show high affinity for both the dopamine D₂ receptor and the serotonin reuptake site. The compounds show activity as antagonists at dopamine D₂ receptors as they potentially antagonize apomorphine-induced climbing behaviour in mice. The compounds also show activity as inhibitors of serotonin reuptake, as they potentiate 5-HTP induced behaviour in mice. The compounds are active in therapeutic models sensitive to clinically relevant antipsychotics (e.g. the conditioned avoidance response; Van der Heyden & Bradford, Behav. Brain Res., 1988, 31:61-67) and antidepressants or anxiolytics (e.g. suppression of stress-induced vocalization; van der Poel et aL, Psycho-pharmacology, 1989, 97: 147-148). In contrast to clinically relevant dopamine D₂ receptor antagonists the described compounds have a low propensity to induce catalepsy in rodents and as such are likely to induce less extrapyramidal side effects than existing antipsychotic agents. The inhibitory activity of serotonin reuptake inherent in these compounds may be responsible for the therapeutic effects observed in behavioural models sensitive to either antidepressants or anxiolytics. The compounds can be used for the treatment of affections or diseases of the central nervous system caused by disturbances in either the dopaminergic or serotonergic systems, for example: aggression, anxiety disorders, autism, vertigo, depression, disturbances of cognition or memory, Parkinson's disease, and in particular schizophrenia and other psychotic disorders.

### GENERAL ASPECTS OF SYNTHESES

The synthesis of all piperazine derivatives in this patent can be performed as depicted in Scheme 1 for the preparation of compound 3.The starting phenyl piperazines can be obtained as described in EP 0 189 612: Hartog, J et al., 1985: ‘New pharmaceutical compositions having a psychtropic activity; Feenstra, R. W.; de Moes, J. P; Hofma, J.; Kling, H.; Kuipers, W; Long, S. K.; Tulp, M. T. M.; Van der Heyden, J. A. M and Kruse, C. G.; ‘New 1-aryl4-(biarylmethylene)piperazines as potential atypical antipsychotics sharing dopamine D₂ receptor and serotonin 5HT_{1A} receptor affinities. Bioorg. & Med. Chem. Lett., 2001, 11, 2345-2349 and WO 01/14330. The alkylphenone derivatives 2 are commercially available.

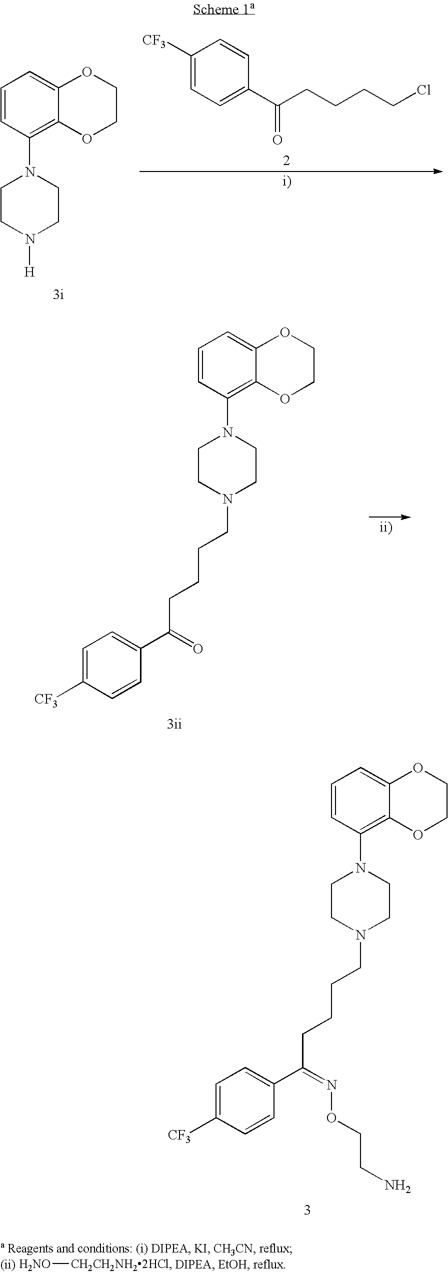

The selection of the particular synthetic procedures depends on factors known to those skilled in the art such as the compatibility of functional groups with the reagents used, the possibility to use protecting groups, catalysts, activating and coupling reagents and the ultimate structural features present in the final compound being prepared.

Pharmaceutically acceptable salts may be obtained using standard procedures well known in the art, for example by mixing a compound of the present invention with a suitable acid, for instance an inorganic acid such as hydrochloric acid, or with an organic acid.

### PHARMACEUTICAL PREPARATIONS

The compounds of the invention can be brought into forms suitable for administration by means of usual processes using auxiliary substances such as liquid or solid carrier material. The pharmaceutical compositions of the invention may be administered enterally, orally, parenterally (intramuscularly or intravenously), rectally or locally (topically). They can be administered in the form of solutions, powders, tablets, capsules (including microcapsules), ointments (creams or gel) or suppositories. Suitable excipients for such formulations are the pharmaceutically customary liquid or solid fillers and extenders, solvents, emulsifiers, lubricants, flavorings, colorings and/or buffer substances. Frequently used auxiliary substances which may be mentioned are magnesium carbonate, titanium dioxide, lactose, mannitol and other sugars, talc, lactoprotein, gelatin, starch, cellulose and its derivatives, animal and vegetable oils such as fish liver oil, sunflower, groundnut or sesame oil, polyethylene glycol and solvents such as, for example, sterile water and mono- or polyhydric alcohols such as glycerol.

Compounds of the present invention are generally administered as pharmaceutical compositions which are important and novel embodiments of the invention because of the presence of the compounds, more particularly specific compounds disclosed herein. Types of pharmaceutical compositions that may be used include but are not limited to tablets, chewable tablets, capsules, solutions, parenteral solutions, suppositories, suspensions, and other types disclosed herein or apparent to a person skilled in the art from the specification and general knowledge in the art. In embodiments of the invention, a pharmaceutical pack or kit is provided comprising one or more containers filled with one or more of the ingredients of a pharmaceutical composition of the invention. Associated with such container(s) can be various written materials such as instructions for use, or a notice in the form prescribed by a governmental agency regulating the manufacture, use or sale of pharmaceuticals products, which notice reflects approval by the agency of manufacture, use, or sale for human or veterinary administration.

Pharmacological Methods

In Vitro Affinity for Dopamine-D₂ Receptors Affinity of the compounds for dopamine-D₂ receptors was determined using the receptor binding assay described by 1. Creese, R. Schneider and S. H. Snyder: “[³H]-Spiroperidol labels dopamine receptors in rat pituitary and brain”, Eur.J.Pharmacol., 46, 377 - 381, 1977.

In vitro Affinity for Serotonin Reuptake Sites

Affinity of the compounds for serotonin reuptake sites was determined using the receptor binding assay described by E. Habert et al.,: “Characterisation of [³H]-paroxetine binding to rat cortical membranes”, Eur.J.Pharmacol., 118, 107 - 114, 1985.

Dosages

The affinity of the compounds of the invention for dopamine-D₂ receptors and serotonine reuptake sites was determined as described above. From the binding affinity measured for a given compound of formula (1), one can estimate a theoretical lowest effective dose. At a concentration of the compound equal to twice the measured Kᵢ-value, 100% of the receptors likely will be occupied by the compound. Converting that concentration to mg of compound per kg of patient yields a theoretical lowest effective dose, assuming ideal bioavailability. Pharmacokinetic, pharmacodynamic, and other considerations may alter the dose actually administered to a higher or lower value. The dosage expediently administered is 0.001-1000 mg/kg, preferably 0.1-100 mg/kg of patient's bodyweight.

Treatment

The term ‘treatment’ as used herein refers to any treatment of a mammalian, preferably human condition or disease, and includes: (1) preventing the disease or condition from occurring in a subject which may be predisposed to the disease but has not yet been diagnosed as having it, (2) inhibiting the disease or condition, i.e., arresting its development, (3) relieving the disease or condition, i.e., causing regression of the condition, or (4) relieving the conditions caused by the disease, i.e., stopping the symptoms of the disease.

The preparation of the compounds having formula (I) will now be described in more detail in the following Examples.

### EXAMPLES

### Example 1

Materials and Methods

¹H and ¹³C NMR spectra were recorded on a Bruker Avance DRX600 instrument (600 MHz), Varian UN400 instrument (400 MHz) or on a Varian VXR200 instrument (200 MHz) using DMSO-D₆ or CDCl₃ as solvents with tetramethylsilane as an internal standard. Chemical shifts are given in ppm (δ scale) downfield from tetramethylsilane. Coupling constants (J) are expressed in Hz. Flash chromatography was performed using silica gel 60 (0.040-0.063 mm, Merck). Column chromatography was performed using silica gel 60 (0.063-0.200 mm, Merck). Melting points were recorded on a Büchi B-545 melting point apparatus. Mass spectra were recorded on a Micromass QTOF-2 instrument with MassLynx application software for acquisition and reconstruction of the data. Exact mass measurement was done of the quasimolecular ion [M+H]⁺.

### Example 2

Syntheses of Specific Compounds

The synthesis of compound 3 is a 2-step reaction starting from 4-(2,3 dihydro-1,4 benzodioxin-5-yl)-1-piperazine (3i). 15 mmol of piperazine (3i) was suspended in 125 ml of acetonitril and 2 equivalents of diisopropylethyl-amine (DIPEA) was added. After 5 minutes stirring at room temperature, 1 equivalent (15 mmol) of 5-chloro-1-(4-trifluoromethyl-phenyl)-pentane-1 -one was added, followed by 1 equivalent of sodium iodide. This mixture was stirred at 80° C. for 20 hours. The solvent was removed by evaporation and the residue dissolved in 100 ml of dichloromethane.

The organic layer was washed with water and dried on magnesium sulphate before evaporation. The residue was purified by column chromatography and this yielded 6.4 mmol of the keto-derivative 3ii which was dissolved in 30 ml of methanol. To this solution 1 equivalent of O-(2-aminoethyl)-hydroxylamine di-HCl salt was added and this mixture was heated for 12 hours at 80° C. After evaporation of the solvent, the residue was dissolved in dichloromethane and washed with water. Drying of the organic layer, using magnesium sulphate and evaporation of the solvent yielded a residue that was purified by column chromatography. The tri HCl-salt of compound 3 was obtained after adding 3 equivalents of HCl in Ethanol to the purified substance. mp.156-60° C.; overall yield 15%.

The synthesis of compound 7 is a 2-step reaction starting from 2-isopropyloxy-phenylpiperazine (7i). 4.2 mmol of phenyl piperazine 7i was suspended in 40 ml acetonitril. Added were 2 eq. of DIPEA, 1 eq. of 4-chloro-1-(4-trifluoromethyl-phenyl)-butane-1-one and 1 eq. potassium iodide. This mixture was refluxed overnight and the solvent evaporated the next day. The residue was purified by column chromatography, yielding 2.5 mmol of the pure keto-compound 7ii, which was dissolved again in 30 ml of ethanol (100%). Added was 1 eq. of 0-(2-aminoethyl) hydroxylamine di-HCl salt and 1 eq. of pyridine. This mixture was heated at 80° C. for 4 hours. After solvent evaporation the residue was purified by column chromatography and this yielded 2 mmol of an orange oil. The oily substance was dissolved in ethanol and added was 1 eq. of fumaric acid. The amorph fumaric salt of compound 7 was obtained after evaporation; overall yield 50%

The synthesis of compound 8 is a 2-step reaction starting from 4-(2,3 dihydro-1,4 benzodioxin-5-yl)-1-piperazine (3i). 3.5 mmol of phenyl piperazine (3i) was suspended in 40 ml acetonitril. Added were 2 eq. of DIPEA, 1 eq. of 6-chloro-1-(4-trifluoromethyl-phenyl)-hexane-1-one and 1 eq. potassium iodide. This mixture was refluxed overnight and the solvent evaporated the next day. The residue was purified by column chromatography, yielding 1.7 mmol of the pure keto-compound 8ii, which was dissolved again in 10 ml of ethanol (100%). Added was 1 eq. of 0-(2-aminoethyl) hydroxylamine di HCl salt and this mixture was heated at 80° C for 4 hours. After solvent evaporation the residue was purified by column chromatography and this yielded 1.6 mmol of an yellow oil. The oily substance was dissolved in ethanol and added was 2 eq. of fumaric acid. The amorph fumaric salt of compound 8 was obtained after evaporation; overall yield 45%.

The synthesis of compound 9 is a 2-step reaction starting from 4-2,3 dihydro-1,4 benzodioxin-5-yl)-1-piperazine. Compound 3ii (30 mmol) was dissolved in 20 ml of methanol. To this solution, 1 equivalent of O-(N-methyl-2-aminoethyl)-hydroxylamine di-HCl salt was added and this mixture was heated for 5 hours at 80° C. After evaporation of the solvent, the residue was dissolved in dichloromethane and washed with sodium bicarbonate solution and followed by brine. The organic layer was dried by using magnesium sulphate and evaporation of the solvent yielded a residue that was purified by column chromatography. The fumaric-salt of the compound 9 was obtained after adding an ethanolic solution of 1 equivalents of fumaric acid to the purified substance followed by evaporation of the solvent; overall yield 15%.

The synthesis of compound 10 is a 2-step reaction starting from 4-(2,3 dihydro-1,4 benzodioxin-5-yl)-1-piperazine (3i). 6 mmol of phenyl piperazine 3i was suspended in 40 ml acetonitril. Added were 2 eq. of DIPEA, 1 eq. of 7-chloro-1-(4-trifluoromethyl-phenyl)-heptane-1-one and 1 eq. potassium iodide. This mixture was refluxed overnight and the solvent evaporated the next day. The residue was purified by column chromato-graphy, yielding 4.2 mmol of the pure keto-compound 10ii, which was dissolved again in 25 ml of ethanol (100%). Added was 1 eq. of O-(2-aminoethyl) hydroxylamine di-HCl salt and this mixture was heated at 80° C. for 4 hours. After solvent evaporation the residue was purified by column chromatography and this yielded 2 mmol of an yellowy oil. The oily substance was dissolved in ethanol and added was 1.5 eq. of fumaric acid. The amorph fumaric salt of compound 10 was obtained after evaporation; overall yield 35%.

TABLE 1
Compounds of the general formula (1)

| | |
|---|---|
| | (1) |
| 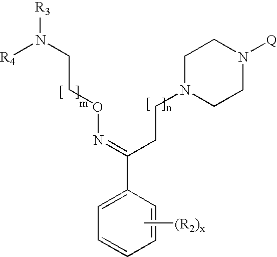 | |

wherein Q can be one of the structural fragments A-N

| | |
|---|---|
| 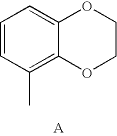 | 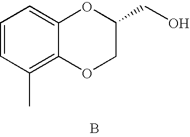 |
| 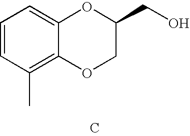 | 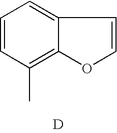 |
| 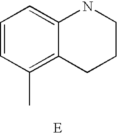 | 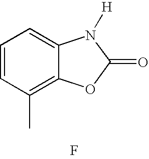 |
| 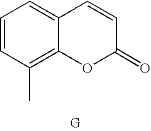 | 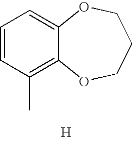 |
| 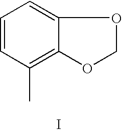 | 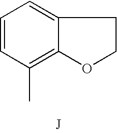 |
| 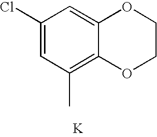 | 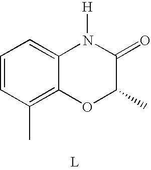 |
| 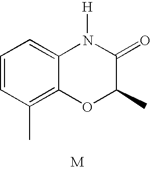 | 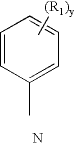 |

| cpnd | y | R₁ | Q | x | R₂ | R₃ | R₄ | n | m | mp(° C.) |
|---|---|---|---|---|---|---|---|---|---|---|
| 3 | — | — | A | 1 | 4- | H | H | 3 | 1 | 156-160 |
| | | | | | CF₃ | | | | | |
| 4 | — | 2-OMe | N | 1 | 4-F | H | H | 2 | 1 | 150-152 |
| 5 | — | — | F | 1 | 4- | H | H | 3 | 1 | 158 |
| | | | | | CF₃ | | | | | |
| 6 | — | — | D | 1 | 4- | H | H | 3 | 1 | 153-154 |
| | | | | | CF₃ | | | | | |
| 7 | 1 | OCH(Me)₂ | N | 1 | 4- | H | H | 2 | 1 | amorph |
| | | | | | CF₃ | | | | | |
| 8 | — | — | A | 1 | 4- | H | H | 4 | 1 | amorph |
| | | | | | CF₃ | | | | | |
| 9 | — | — | A | 1 | 4- | Me | H | 3 | 1 | amorph |
| | | | | | CF₃ | | | | | |
| 10 | — | — | A | 1 | 4- | H | H | 5 | 1 | amorph |
| | | | | | CF₃ | | | | | |
| 11 | 1 | 2-OMe | N | 1 | 4- | H | H | 5 | 1 | amorph |
| | | | | | CF₃ | | | | | |

The specific compounds of which the synthesis is described above are intended to further illustrate the invention in more detail, and therefore are not deemed to restrict the scope of the invention in any way. Other embodiments of the invention will be apparent to those skilled in the art from consideration of the specification and practice of the invention disclosed herein. It is thus intended that the specification and examples be considered as exemplary only, with a true scope and spirit of the invention being indicated by the claims.

### Example 3

Formulation of Comp. 3 Used in Animal Studies

For oral (p.o.) administration: to the desired quantity (0.5-5 mg) of the solid compound 3 in a glass tube, some glass beads were added and the solid was milled by vortexing for 2 minutes. After addition of 1 ml of a solution of 1% methylcellulose in water and 2% (v/v) of Poloxamer 188 (Lutrol F68), the compound was suspended by vortexing for 10 minutes. The pH was adjusted to 7 with a few drops of aqueous NaOH (0.1 N). Remaining particles in the suspension were further suspended by using an ultrasonic bath.

For intraperitoneal (ip.) administration: to the desired quantity (0.5-15 mg) of the solid compound 3 in a glass tube, some glass beads were added and the solid was milled by vortexing for 2 minutes. After addition of 1 ml of a solution of 1% methylcellulose and 5% mannitol in water, the compound was suspended by vortexing for 10 minutes. Finally the pH was adjusted to 7.

### Example 4

Pharmacological Testresults

Dopamine-D₂ and serotonin reuptake receptor affinity data obtained according to the protocols given above are shown in the table below.

TABLE 2
In vitro affinities of compounds of the invention

In vitro affinity

| | Dopamine-D₂ | 5-HT reuptake |
|---|---|---|
| cpnd | pKᵢ | pKᵢ |
| 3 | 8.3 | 8.2 |
| 4 | 8.6 | 7.3 |
| 5 | 8.2 | 8.3 |
| 6 | 6.6 | 8.3 |
| 7 | 8.1 | 7.0 |
| 8 | 8.3 | 8.0 |
| 9 | 8.4 | 7.5 |
| 10 | 8.4 | 8.5 |
| 11 | 8.3 | 8.3 |

1. Compounds of the general formula (1): 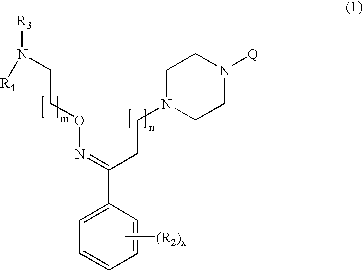
wherein:
m and n independently are either 1, 2, 3, 4, 5, 6, 7 or 8,
x is 0, 1, 2 or 3
R₂ is halogen, branched or unbranched alkyl(C₁₋₆), phenyl, benzyl, branched or unbranched alkoxy(C₁₋₆), trifluoromethyl or cyano
R₃ and R₄ independently represent hydrogen, alkyl(C₁₋₆), phenyl, benzyl or acetyl
group Q is chosen from structural fragments A—N 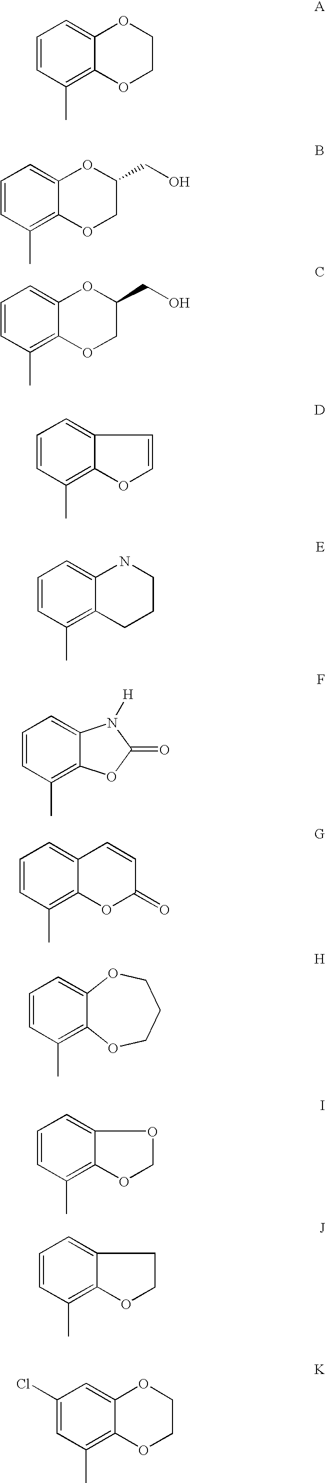 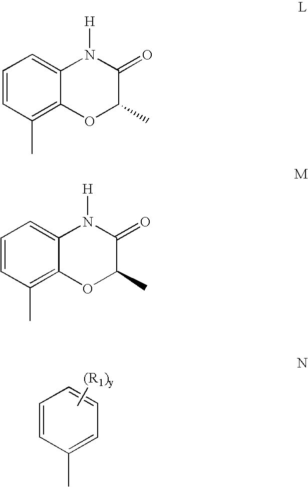
wherein:
y is 1, 2 or 3
R₁ is halogen, branched or unbranched alkyl(C₁₋₆), phenyl, benzyl, branched or unbranched alkoxy(C₁₋₆), trifluoromethyl or cyano,
and tautomers, stereoisomers and N-oxides thereof, as well as pharmacologically acceptable salts, hydrates and solvates of said compounds of formula (1) and its tautomers, stereoisomers and N-oxides.

2. Compounds as claimed in claim 1 of general formula (1) wherein m is 1, n is 2, 3, 4 or 5, x is 1, R₂ is 4-fluoro or 4-trifluoromethyl, R₃ and R₄ independently represent hydrogen or methyl, group Q is chosen from structural fragments A, D, F or N, y is 1, R₁ is branched or unbranched alkoxy(C₁₋₃), and tautomers, stereoisomers and N-oxides thereof, as well as pharmacologically acceptable salts, hydrates and solvates of said compounds of formula (1) and its tautomers, stereoisomers and N-oxides.

3. A compound as claimed in claim 1, selected from the group:

| y | R₁ | Q | x | R₂ | R₃ | R₄ | n | m |
|---|---|---|---|---|---|---|---|---|
| — | — | A | 1 | 4-CF₃ | H | H | 3 | 1 |
| 1 | 2-OMe | N | 1 | 4-F | H | H | 2 | 1 |
| — | — | F | 1 | 4-CF₃ | H | H | 3 | 1 |
| — | — | D | 1 | 4-CF₃ | H | H | 3 | 1 |
| 1 | OCH(Me)₂ | N | 1 | 4-CF₃ | H | H | 2 | 1 |
| — | — | A | 1 | 4-CF₃ | H | H | 4 | 1 |
| — | — | A | 1 | 4-CF₃ | Me | H | 3 | 1 |
| — | — | A | 1 | 4-CF₃ | H | H | 5 | 1 |
| 1 | 2-OMe | N | 1 | 4-CF₃ | H | H | 5 | 1 |

wherein the symbols represent those in formula (1): 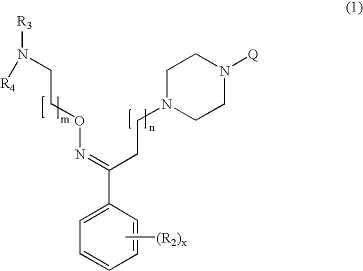
and the structural fragments A, D, F or N: 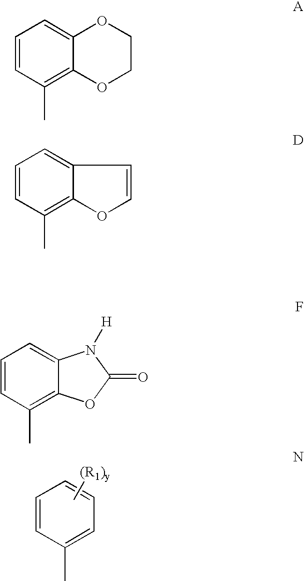
and tautomers, stereoisomers and N-oxides thereof, as well as pharmacologically acceptable salts, hydrates and solvates of said compounds of formula (1) and its tautomers, stereoisomers and N-oxides.

4. A pharmaceutical composition comprising, in addition to a pharmaceutically acceptable carrier and/or at least one pharmaceutically acceptable auxiliary substance, a pharmacologically active amount of at least one compound of one of the claims 1-3, or a salt thereof, as an active ingredient.

5. A method of preparing a composition as claimed in claim 4, characterised in that at least one compound of one of the claims 1-3, or a salt thereof, is brought into a form suitable for administration.

6. A compound as claimed in any of the claims 1-3, or a salt thereof, for use as a medicament

7. Use of a compound as claimed in any of the claims 1-3 for the preparation of a pharmaceutical composition for the treatment of CNS disorders.

8. Use as claimed in claim 7, characterized in that said disorders are aggression, anxiety disorders, autism, vertigo, depression, disturbances of cognition or memory, Parkinson's disease, schizophrenia and other psychotic disorders.

9. Use as claimed in claim 7, characterized in that said disorder is depression.

10. Use as claimed in claim 7, characterized in that said disorders are schizophrenia and other psychotic disorders.
